(19) United States
(12) Patent Application Publication
(10) Pub. No.: US 2019/0175953 A1
(43) Pub. Date: Jun. 13, 2019

(54) CARE PROTOCOL FOR REDUCING LONG AND SHORT-TERM ADVERSE EFFECTS CAUSED BY RADIOTHERAPY OR RADIOSURGERY TREATMENT

(71) Applicant: CONVERGENT R.N.R LTD, Tirat Carmel (IL)

(72) Inventors: Aharon BAR-DAVID, NESHER (IL); Miri MARKOVICH, HAIFA (IL); Zeev BURSHTEIN, NES-ZIONA (IL); Zeev HAREL, KFAR SABA (IL)

(73) Assignee: CONVERGENT R.N.R LTD, TIRAT CARMEL (IL)

(21) Appl. No.: 16/258,612

(22) Filed: Jan. 27, 2019

Related U.S. Application Data
(63) Continuation-in-part of application No. 15/102,713, filed on Jun. 8, 2016, now abandoned.
(60) Provisional application No. 61/913,937, filed on Dec. 10, 2013.

(51) Int. Cl. A61N 5/10

(52) U.S. Cl. CPC A61N 5/1084; A61N 5/1065; A61N 5/1069; A61N 5/1031

(57) ABSTRACT

A method of a radiotherapy or radiosurgery treatment comprises steps of: (a) providing a converging x-ray beam source configured for emitting a converging X-ray beam propagating along an axis thereof; (b) emitting the converging x-ray beam towards a volume of treatment (VOT) having a length along the axis of the converging X-ray beam ranging between 2 mm and 5 cm within a patient's body such that a waist portion is within the VOT; (c) propagating the beam through tissues previously located relative to the VOT (PO); the VOT per se and tissues distally located to the VOT (DO). The converging X-ray beam characterized by a convergent angle ranging between 2 and 30 degrees providing 80% to 100% of a maximum dose is received by the VOT and less than 60% of the maximum dose is received by the PO and the DO.

### CROSS REFERENCE TO RELATED APPLICATIONS

This application is a Continuation-in-Part of application Ser. No. 15/102,713 filed Jun. 8, 2016, which is a U.S. National Stage Entry of PCT International Application Number PCT/IL2014/051065 filed Jul. 12, 2014, which claims priority from provisional application No. 61/913,937 filed Oct. 12, 2013. All of these applications are hereby incorporated by reference in their entirety.

### FIELD OF THE INVENTION

The current invention pertains to increased effectiveness and reduced adverse effects of a radiotherapy or radiosurgery treatment and more particularly to a care protocol for using a converging x-ray beam.

### BACKGROUND OF THE INVENTION

Radiation therapy (radiotherapy) uses high-energy ionizing radiation to control tumors, kill cancer cells and prevent their recurrence. About 60% of cancer cases require radiation therapy while the most common types of cancer treated that way are prostate, skin, head and neck, throat, larynx, breast, brain, colon-rectal, lung, bone, leukemia, ovarian, and uterine. In some cases, radiotherapy is combined with chemotherapy and/or surgical removal of the cancerous tumor. In any case a radiotherapy or radiosurgery treatment plan is made.

The goal of a treatment plan is to target the radiation to the tumor with minimal effect on the surrounding healthy tissue. The plan is contemplated according to simulations on the patient's inner body imaging data, which are used to plan the geometric, radiological, and dosimetric aspects of the therapy using radiation transport simulations and optimization. Plans are often assessed with the aid of dose-volume histograms (DVH), allowing the clinician to evaluate the uniformity of the dose to the diseased tissue (tumor) and sparing of healthy structures.

In all inner-body radiotherapy or radiosurgery treatment s, radiation passes through healthy tissue on its way to and from the volume of the patient's body that is under treatment causing various adverse effects. The main reported adverse effects are fatigue and skin irritation. Additional short-term adverse effects are: nausea and vomiting, damage to the epithelial diseased tissue, mouth and throat sores, intestinal discomfort, swelling, infertility and various other adverse effects of different amount of severity. Long-term effects are: fibrosis, dryness, lymphedema, secondary cancer, heart disease, cognitive decline and radiation proctitis.

In order to reduce damage to healthy tissue in radiation therapy and to prevent adverse effects caused by it, different delivery systems of radiation that reduce their exposure to healthy tissue were suggested. For example, brachytherapy places a radiation source in or next to the volume requiring therapy. For example, US2004116767 patent application suggests a device for providing radiation to treat breast cancer, and U.S. Pat. No. 6,200,255 patent offers a device for delivering radiotherapy to the prostate gland. The major disadvantage of brachytherapy is that it requires an invasive procedure, which many patients, already very ill, will not be able to tolerate.

Today's radiotherapy and radiosurgery treatments are done using linear accelerators (LINAC). The main disadvantages of LINACs include: Non-converging (even diverging) beams, which causes the need to scan the body from many directions: use of high energy photons in the range of a few MeV up to about 25 MeV, which causes the beam to be only little attenuated after it passes the volume of treatment (VOT): high cost of the machine and its accessories and more.

There thus remains a long felt need for a standard of care protocol for radiotherapy or radiosurgery treatments that will improve the quality of the treatment, reduce substantially the number of sessions needed as well as reduce the amount and severity of adverse effects without requiring an invasive procedure. It is important to reduce long-term effects, but nonetheless, it is important to reduce the short-term effects as well. The latter, though not life threatening, add to the stress and anxiety and may interfere with the heeling progression.

### SUMMARY OF THE INVENTION

It is hence one object of the invention to disclose a method of a radiotherapy or radiosurgery treatment. The aforesaid method comprises steps of: (a) providing a converging x-ray beam source configured for emitting a converging X-ray beam propagating along an axis thereof; (b) emitting the converging x-ray beam towards a volume of treatment (VOT) having a length along the axis of the converging X-ray beam ranging between 2 mm and 5 cm within a patient's body such that a waist portion is within the VOT; (c) May be propagating the beam through at least one organ previously located relative to the VOT (PO); the VOT per se and may pass at least one organ distally located to the VOT (DO).

It is a core purpose of the invention to provide the converging X-ray beam characterized by a convergent angle ranging between 2 and 30 degrees providing at least 80 to 100% of a maximum dose received by the VOT and less than 60% of the maximum dose received by the PO and the DO in one shot from a single direction.

Another object of the invention is to disclose a method of a radiotherapy or radiosurgery treatment. The aforesaid method comprises steps of: (a) providing a converging x-ray beam source configured for emitting a converging X-ray beam propagating along an axis thereof; (b) emitting the converging x-ray beam towards a volume of treatment (VOT) having a length along the axis of the converging X-ray beam ranging between 2 mm and 5 cm within a patient's body such that a waist portion is within the VOT; (c) propagating the beam through the VOT per se and may pass at least one organ distally located to the VOT (DO).

It is a core purpose of the invention to provide the converging X-ray beam characterized by a convergent angle ranging between 2 and 30 degrees providing at least 80 to 100% of a maximum dose received by the VOT and less than 60% of the maximum dose received by the DO in one shot from a single direction.

### BRIEF DESCRIPTION OF THE FIGURES

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not necessarily obscured. In the accompanying drawing:

FIG. 1 schematically illustrates a radiotherapy or radiosurgery treatment with a converging x-ray beam source;

FIG. 2 is a 2-dimensional (2D) grey-scale dose distribution presentation of a longitudinal cross-section of the center of a single shot of a converging x-ray beam;

FIG. 3 is an illustration of the percentage of the dose distribution as function of depth of penetration, which is called: Percentage Depth Dose (PDD) of different types of x-ray beams;

FIG. 4 is a schematic diagram illustrating variable parameters of a radiotherapy or radiosurgery treatment; and

FIGS. 5a and 5b are exemplary graphs of maximal Percentage Depth Dose (mPDD) corresponding to different diverging angle α.

### DETAILED DESCRIPTION OF THE PREFERRED EMBODIMENTS

The following description is provided, alongside all chapters of the present invention, so as to enable any person skilled in the art to make use of the invention and set forth the best modes contemplated by the inventor of carrying out this invention. Various modifications, however, will remain apparent to those skilled in the art, since the generic principles of the present invention is defined to specifically provide a standard of care protocols for radiotherapy or radiosurgery treatments utilizing a converging x-ray beam source that increases treatment's efficiency and reduces the amount of acute and long term adverse effects.

The term “single shot” refers to a single shot in a single direction.

The term “radiotherapy” refers hereinafter to the medical use of ionizing radiation, generally as part of cancer treatment to control or kill malignant cells. It may also be used as part of adjuvant therapy, to prevent tumor recurrence after surgery that removes a primary malignant tumor. Radiation therapy may be synergistic with chemotherapy, and may be used before, during, and after chemotherapy in susceptible cancers. According one embodiment, radiotherapy relates to a mode of treatment wherein the therapeutic dose can be administered in more than one fraction, usually in a number of fractions, for example in more than 10 fractions.

The term “adverse effects” refers hereinafter to a harmful and undesired effect resulting from the radiotherapy or radiosurgery treatment.

The term “short-term adverse effects” refers hereinafter to varying side effects occurring during the treatment course and a short period of time after them. For example, the term refers, to fatigue, skin irritation, nausea and vomiting, damage to the epithelial surfaces, mouth and throat sores, intestinal discomfort, swelling and infertility.

The term “long-term adverse effects” refers hereinafter to varying side effects appearing months or years following treatment. For example, the term refers to fibrosis, epilation, dryness, lymphedema, secondary cancer, heart disease and cognitive decline.

The term “converging x-ray beam” refers to a beam whose rays start from separate spread locations and converging to a common location—the focal location—at the focal distance. It can be a point—focal point, or small cross section area at the focal plane, the waist of the converging beam. Thus, the average radiation flux cross section area density is increasing along the longitudinal axis until reaching a maximum related to the focal location. Beyond the focal distance the rays diverge. The convergence of the x-ray beam improves the quality of the treatment, reduces substantially the number of sessions needed as well as reduces the amount and severity of adverse effects without requiring an invasive procedure.

The term “secondary cancer” refers hereinafter to cancer caused by cell damage resulting from radiotherapy or chemotherapy.

The term “bodily cavity” refers herein after to a natural hollow or sinus within the body. More specifically the term refers to the oral cavity, anal cavity, vagina, nasal cavity, ear cavity, eye socket, etc.

The term “internal organ” refers herein after to an organ that is situated inside the body. More specifically, the term relates to the heart, kidney, lungs, liver, womb, bone and any other organ within the body.

The term “radiosurgery” refers hereinafter to a regime of treatment with fewer sessions than the number of treatments used in radiotherapy, of a much higher dose in each session.

The term “volume of treatment (VOT)” refers hereinafter to the volume of the treated target. This term usually refers to a cancerous tumor.

The term “preceding organ (PO)” refers hereinafter to an organ that precedes the VOT in the trajectory of the x-ray beam. More generally, the term refers, for example, to skin, brain, liver, kidney, heart, lungs, etc.

The term “distal organ (DO)” refers hereinafter to an organ that follows the VOT in the trajectory of the x-ray beam. More generally, the term refers, for example, to skin, brain, liver, kidney, heart, lungs, etc.

The term “focal point” refers hereinafter to the point at which the converging x-ray beams meet.

The term “maximum dose” refers hereinafter to the highest dose absorbed in a complete treatment (several directions) or in a single shot from a single direction in the case of a converging beam.

The term “maximum dose location” refers herein after to the location or locations where the maximum dose is received.

The term “dose volume histogram (DVH)” refers hereinafter to a concept used in radiation treatment planning to summarize three-dimensional (3D) dose distributions in a graphical two-dimensional (2D) format.

The term “cumulative dose volume histogram (cDVH)” refers hereinafter to a dose volume histogram plotted with bin doses along the horizontal axis and the column height of each bin represents the volume of structure receiving greater than or equal to the dose that belongs to that bin. With very fine bin sizes, the cDVH takes on the appearance of a smooth line graph. The lines always slope and start from top-left to bottom-right.

The term “differential dose volume histogram (dDVH)” refers hereinafter to a dose volume histogram plotted with bin doses along the horizontal axis where the column height of each bin represents the volume of structure receiving the dose equal to the dose that belongs to that bin (which represents the bin center)±the amount of half a bin width. With very fine bin sizes, the dDVH takes on the appearance of a smooth line graph. The DVH is utilized for determining TCI, PITY, TVR, HI, DGI, TCP, NTCP

The term “target coverage index (TCI)” refers hereinafter to an index describing the exact coverage of the target volume in a treatment plan at a given prescription dose as expressed below:

$$TCI = \frac{{PTV}_{PD}}{PTV} ,$$

where PTV refers to the planned target volume (taking into account the machine limitation) and PTV_{PD} refers to the prescribed dose target volume. Ideally TCI=1, meaning that the planned prescribed dose equals the planned target volume which takes into account the limitations of the machine, as expressed below:

The term “prescription isodose target volume conformal index (PITY)” refers hereinafter to an index that assesses the conformity of a treatment plan:

$$PITV = \frac{PIV}{PTV} ,$$

where PTV refers to the planned target volume (taking into account the machine limitation) whereas PIV refers to the prescription isodose volume coverage for the target and normal tissues. Good uniformity or how well the prescription isodose line conforms to the size and shape of the planning target volume means that the value should be close to 1 as much as possible.

Sometimes the inverse is defined:

$$TVR = \frac{PTV}{PIV} ,$$

The term “homogeneity index (HI)” refers hereinafter to an index that scales the “hot” spots in and around the planning target volumes, as expressed below:

$$TCI = \frac{{D}_{Max}}{PD} ,$$

where D_{Max} is the maximum dose point in the PTV and PD is the prescribed dose. The higher the homogeneity is the closer the value of HI is to 1.

The term “dose gradient index (DGI)” refers hereinafter to a scale that examines the steepness or shallowness of dose falloff in target volume, as expressed below:

$$TCI = \frac{{PTV}_{PD}}{{PTV}_{0.50 PD}} ,$$

where PTV_{PD} refers to the planned target volume at the prescribed dose, whereas PTV_{0.50PD} is the planning target volume coverage at 50% of PD. Large gradient means less dose to the close surrounding tissues.

The term “normal tissue complication probability (NTCP)” is a probability value that is a function of dose and it refers hereinafter to an index reflecting the probability for complications on normal tissues as a function of dose. The NTCP should be as low as possible.

The term “Tumor control probability (TCP)” is a probability value that is a function of dose and it refers hereinafter to the probability of effectively killing tumor clonogens (tumor cells) as a function of dose taking into account the survival time and proliferation rate of the tumor cells. TCP should be as high as possible.

The term “Wong-Baker FACES™ Pain Rating Scale” refers hereinafter to a scale that shows a series of faces ranging from a happy face at 0, “No hurt”, to a crying face at 10 “Hurts worst” and the patient must choose the face that best describes how they are feeling. The name of the scale is a Trademark.

The term “Bristol Stool Scale” refers hereinafter to medical aid designed to classify the form of human feces into seven categories. It is in use as a research tool to evaluate the effectiveness of treatments for various diseases of the bowel, as well as a clinical communication aid.

The term “steroids” refers hereinafter to a class of chemicals that control carbohydrate, fat and protein metabolism and are anti-inflammatory by preventing phospholipid release, decreasing eosinophil action and a number of other mechanisms.

The term “Fatigue Impact Scale” refers hereinafter to a detailed and relatively lengthy tool, which takes about 3 min to complete in a non-fatigued person, but may take much longer in a severely fatigued respondent. The score reflects functional limitation due to fatigue experienced within the previous month rather than a measure of the level of fatigue.

The term “Graphic Scoring Scale for Nausea” refers hereinafter to a pictorial nausea scale of 0 to 10 with 6 faces. The scale has converging and discriminant validity, along with an ability to detect change after treatment.

The term “Radiotherapy Categorical Anxiety Scale” refers hereinafter to a quantitatively measure for specific types of anxiety among cancer patients receiving radiotherapy.

The term “photochemical reflectance index” refers hereinafter to an index derived from narrow band reflectance at 530 and 570 nm which is an indicator of photosynthetic radiation use efficiency.

The term “erythema” refers hereinafter to redness of the skin, caused by hyperemia of the capillaries in the lower layers of the skin. It occurs with any skin injury, infection, or inflammation.

The term “Self-Rating Scale for Evaluating Memory in Everyday Life” refers hereinafter to a scale for assessing frequency of occurrence of memory failures, and 4 global rating items assessing overall comparison to others, comparison to the best one's memory has ever been, speed of recall, and concern or worry over memory function.

The term “whole body effective dose” refers hereinafter to a radiation type weighted and tissue-weighted sum of the equivalent doses in all specified tissues and organs of the body as defined below:

Calculating from the Equivalent Dose:

E=Σ_{T}W_{T}·H_{T}=Σ_{T}W_{T}Σ_{R}W_{R}·D̅_{T,R}.

Calculating from the Absorbed Dose:

$$E = {Σ}_{T} {W}_{T} {Σ}_{R} {W}_{R} \cdot \frac{{\int }_{T} {D}_{R} ( x , y , z ) ρ ( x , y , z ) dV}{{\int }_{T} ( x , y , z ) ρ ( x , y , z ) dV } ,$$

### Where

E is the whole body effective dose to the entire organism;
H_{T} is the equivalent dose absorbed by tissue T;
W_{T} is the tissue weighting factor defined by regulation;
W_{R} is the radiation weighting factor defined by regulation;
D̅_{T,R} is the mass-averaged absorbed dose in tissue T by radiation type R;
D_{R}(x,y,z) is the absorbed dose from radiation type R as a function of location;
ρ(x,y,z) is the density as a function of location;
V is volume; and
T is the tissue or organ of interest.

A convergent beam is provided by an X-ray source combined with an X-ray lens arrangement disclosed in U.S. Pat. No. 9,008,271, WO2016/108235 and WO201/9003229 which are incorporated by reference in entirety.

Presentation of a received dose at each depth by means of maximal percentage depth dose (mPDD) is disclosed in PCT publication WO2017/141245 which is incorporated by reference in its entirety.

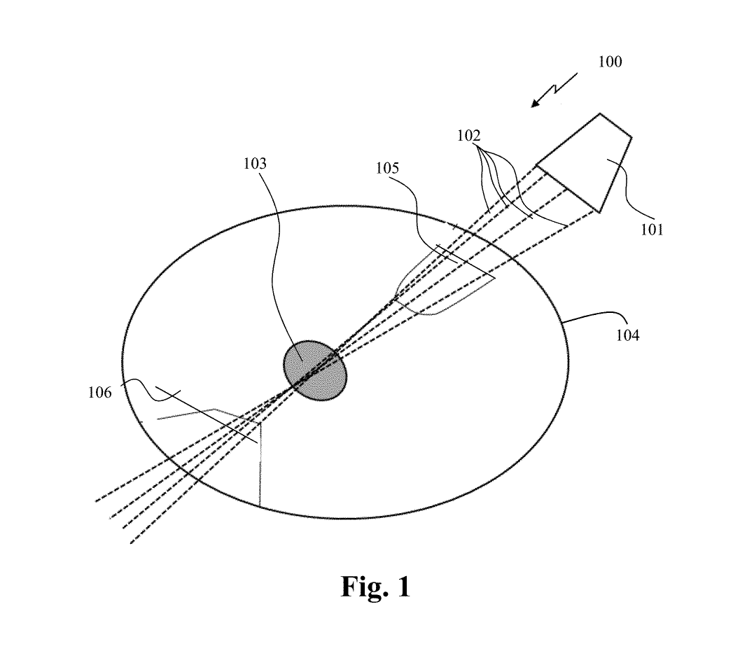

Reference is now made to FIG. 1, schematically illustrating a radiotherapy or radiosurgery treatment utilizing a converging x-ray beam source (100). The converging x-ray beam source (101) emits a converging x-ray beam (102). The beam is targeted towards a volume of treatment (VOT) (103) which is usually an organ infected with cancer within a patient's body (104). Before reaching the VOT the beam may travels through other organs preceding the VOT (105). After exiting the VOT the beam may travels through organs distal to the VOT (106). The converging x-ray beam source enables the VOT to receive a large dosage while the distal and preceding organs receive minimal dosage. Reducing the dosage to healthy tissue reduces the amount of side effects that the patient will suffer from. Therefore, using a converging beam source will reduce long term as well as short term side effects. The increased dose supplied to the VOT will increase the efficiency of the therapy and might even shorten the therapy course making it more tolerable to patients.

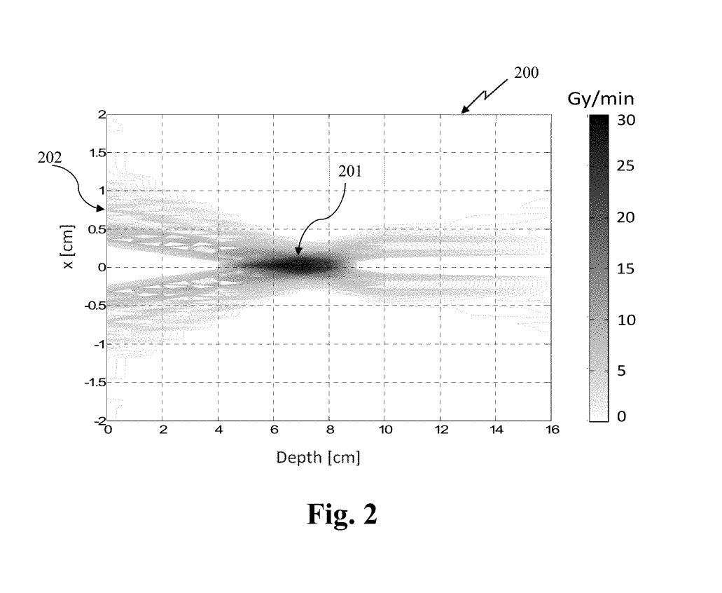

Reference is now made to FIG. 2, a 2-dimensional (2D) dose distribution presentation (200) of a single shot of a converging x-ray beam shown along a longitudinal cross-section at the center of the beam. In this example, the maximum dose at the tumor location (201) of over than 30 Gy/min is given at depth of 6-8 cm and with a transverse cross-section area of about 0.25 cm diameter at the center. The rest of the dose is diverged around this area and does not exceed about an average of 5 Gy/min. In this example the radiation penetrates the skin from the left (202). This shows that the beam can be concentrated in one specific area that is aimed at the volume of treatment (VOT) (201). The VOT is usually a cancerous tumor. The higher the dosage in this area the more successful the treatment is. The rest of the dose around is absorbed by healthy tissue and as result damages it. The lower the radiation is in these areas less adverse effects results from the treatment. The converging beam enable high dose to the VOT while relatively low doses to the area surrounding the VOT and therefore the treatment with this beam is more successful and causes less adverse effects.

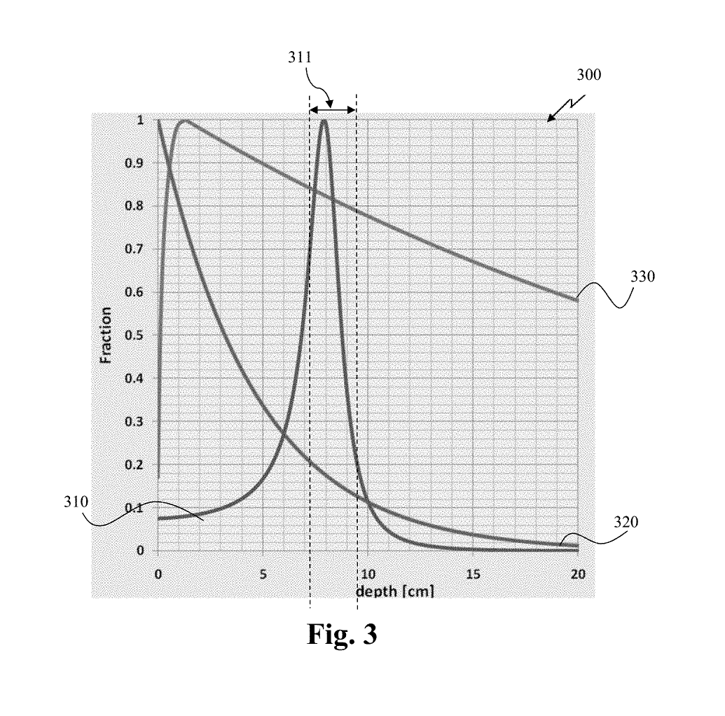

Reference is now made to FIG. 3, an illustration of the percentage of the dose distribution as function of depth of penetration, which is called: Percentage Depth Dose (PDD) of different types of x-ray beams (300). The converging x-ray beam (310) distributes mainly in one region (311) resulting in very high dosage to a specific area while the surroundings of this areas receive much lower dosage. The two other beams, parallel beam from an orthovoltage X-ray source (320) and from a linear accelerator (330) distribute differently. They both smear on a very large area delivering high dosage close to the skin with no peak. When treating a cancerous tumor by radiotherapy or radiosurgery, it is very important that the beam will concentrate on a very specific volume while the surrounding volumes receive as little as possible of the beam. Therefore, the converging beam seems to be much more suitable for radiotherapy or radiosurgery treatments.

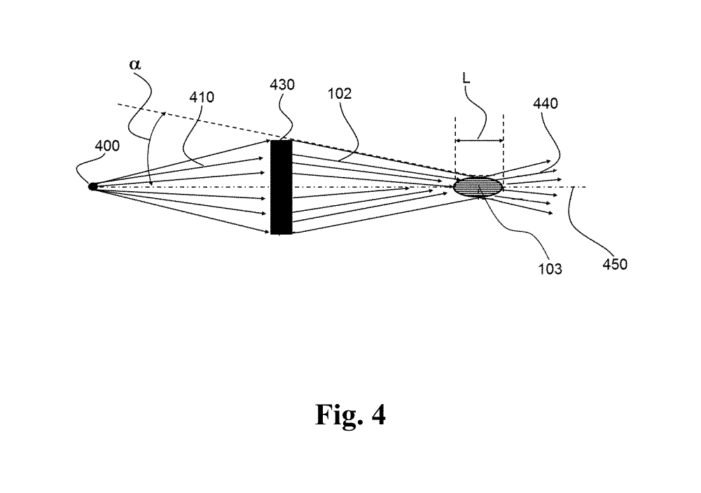

Reference is now made to FIG. 4 presenting a schematic diagram illustrating variable parameters of the convergent beam radiosurgery treatment. X-ray source 400 emits diverging X-ray beam 410 towards lens 430 which converts beam 410 into converging beam 102. The aforesaid beam 102 is characterized by convergent beam angle α ranging from 2° and 30°. This optical arrangement is adapted for tumor 103 characterized by length L measured along axis 450 of convergent beam in the range between 2 mm and 5 cm. Numeral 450 refers to X-rays passing through tumor 103.

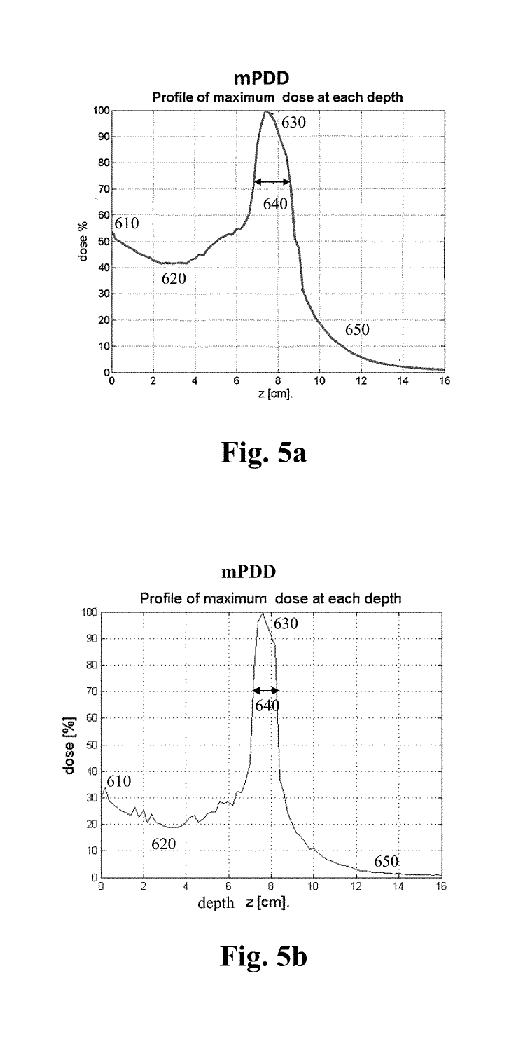

Reference is now to FIGS. 5a and 5b presenting profiles of maximal percentage depth dose (mPDD) corresponding to the convergent beam characterized by converging angles 2° and 30°, respectively. Location 610 at z=0 cm corresponds to a relative dose on the skin where the X-ray beam enters the patient's body. Zones 620 and 650 of the maximal percentage depth dose curves relate to X-ray doses received by tissues located before and after the tumor (volume of treatment) along the direction of beam propagation. Maximal percentage depth dose in zone 630 corresponds to the dose received by the volume of treatment. Numeral 640 refers to full width at half maximum of dose peak at treatment location.

1. A method of a radiotherapy or radiosurgery treatment; said method comprising:
a. providing a converging x-ray beam source configured for emitting a converging X-ray beam propagating along an axis thereof;
b. emitting said converging x-ray beam towards a volume of treatment (VOT) having a length along said axis of said converging X-ray beam ranging between 2 mm and 5 cm within a patient's body such that a waist portion is within said VOT;
c. propagating said beam through previously located tissues relative to said VOT (PO); said VOT per se and tissues distally located to said VOT (DO);
wherein said converging X-ray beam is characterized by a convergent angle ranging between 2 and 30 degrees providing at least 80 to 100% of a maximum dose received by said VOT and less than 60% of said maximum dose received by said PO and said DO in one shot from a single direction.

2. A method of a radiotherapy or radiosurgery treatment; said method comprising:
a. providing a converging x-ray beam source configured for emitting a converging X-ray beam propagating along an axis thereof;
b. emitting said converging x-ray beam towards a volume of treatment (VOT) having a length along said axis of said converging X-ray beam ranging between 2 mm and 5 cm within a patient's body such that a waist portion is within said VOT;
c. propagating said beam through said VOT per se and tissues distally located to said VOT (DO);
wherein said converging X-ray beam is characterized by a convergent angle ranging between 2 and 30 degrees providing at least 80 to 100% of a maximum dose received by said VOT and less than 60% of said maximum dose received by said DO in one shot from a single direction.
